(19) United States
(12) Patent Application Publication
(10) Pub. No.: US 2009/0035823 A1
(43) Pub. Date: Feb. 5, 2009

(54) Ligation-based synthesis of oligonucleotides with block structure

(76) Inventors: Aleksey Soldatov, Berlin (DE); Tatiana Borodina, Berlin (DE); Hans Lehrach, Berlin (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/553,104

(22) PCT Filed: Apr. 14, 2004
(86) PCT No.: PCT/EP04/03921

§ 371 Date: Jul. 13, 2006

(30) Foreign Application Priority Data
Apr. 15, 2003 (EP) 03008653.2

(51) Int. Cl. C12P 19/34; C07H 21/00

(52) U.S. Cl. 435/91.2; 536/25.3; 536/25.32; 435/91.52

(57) ABSTRACT

The present invention relates to a method of producing single-stranded nucleic acid molecules from oligo- or polynucleotides wherein each of said oligo- or polynucleotides has a predefined 5′ or 3′ terminus, comprising the steps of (a) annealing an adaptor oligonucleotide simultaneously or step by step to (aa) a first oligo- or polynucleotide; and (ab) a second oligo- or polynucleotide wherein the 5′-terminus of said adaptor oligonucleotide is complementary in sequence to the 5′ terminus of said first oligo- or polynucleotide and the 3′terminus of said adaptor molecule is complementary in sequence to the 3′ terminus of said second oligo- or polynucleotide; and optionally (a′) simultaneously with or subsequently to step (a) annealing at least one further adaptor oligonucleotide to free termini of said first or second oligonucleotides and to free termini of further oligo- or polynucleotides; (b) optionally filling in gaps between the neighbouring ends of said oligo- or polynucleotides; (c) ligating said oligo- or polynucleotides; and (d) removing said at least one adaptor oligonucleotide. In a preferred embodiment of the method of the invention, said single-stranded nucleic acid molecules represent a collection of nucleic acid molecules wherein either said first or said second oligo- or polynucleotide is invariable in sequence between all members of said collection of nucleic acid molecules.

The present invention relates to a method of producing single-stranded nucleic acid molecules from oligo- or polynucleotides wherein each of said oligo- or polynucleotides has a predefined 5′ or 3′ terminus, comprising the steps of (a) annealing an adaptor oligonucleotide simultaneously or step by step to (aa) a first oligo- or polynucleotide; and (ab) a second oligo- or polynucleotide wherein the 5′-terminus of said adaptor oligonucleotide is complementary in sequence to the 5′ terminus of said first oligo- or polynucleotide and the 3′-terminus of said adaptor molecule is complementary in sequence to the 3′ terminus of said second oligo- or polynucleotide; and optionally (a′) simultaneously with or subsequently to step (a) annealing at least one further adaptor oligonucleotide to free termini of said first or second oligonucleotides and to free termini of further oligo- or polynucleotides; (b) optionally filling in gaps between the neighbouring ends of said oligo- or polynucleotides; (c) ligating said oligo- or polynucleotides; and (d) removing said at least one adaptor oligonucleotide. In a preferred embodiment of the method of the invention, said single-stranded nucleic acid molecules represent a collection of nucleic acid molecules wherein either said first or said second oligo- or polynucleotide is invariable in sequence between all members of said collection of nucleic acid molecules.

The invention is particularly efficient for the synthesis of long polynucleotides or sets of oligonucleotides with block structure.

As is known in the art, oligonucleotide-producing companies cannot guarantee a quantitative yield for oligos longer than 100 nucleotides (nt). Moreover, the yield and quality of the synthesis decrease dramatically when the length of oligonucleotide is more than 60 nt. The smallest possible scale of synthesis for 80-100 mers is more then 200 nmol. Two-step purification (HPLC and PAGE) is required to obtain single-band oligonucleotides. The guaranteed output of purified 80-100 mers is less than 1 nmol and the price is about 200-300 Euro.

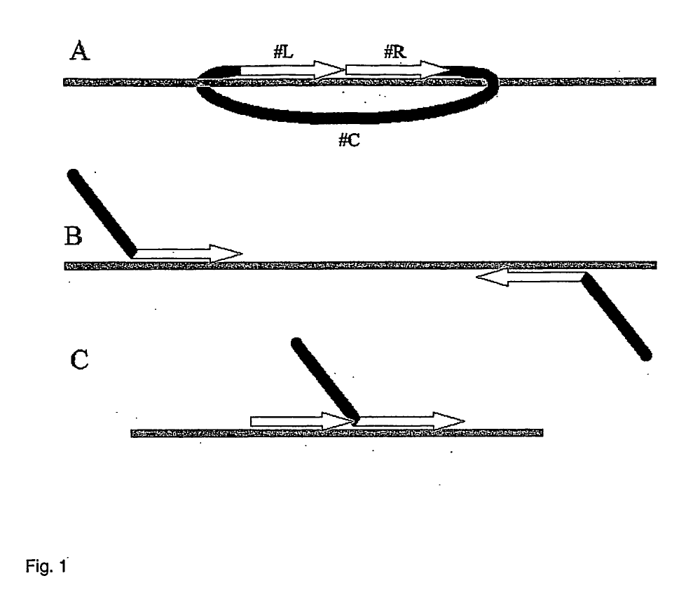

Oligonucleotides with block structure are widely used in molecular biology (FIG. 1). Examples are: padlock probes (Lizardi et al. 1998; Pickering et al. 2002); primers with constant 5′ regions, used for multiplex PCR amplification (Favis et al. 2000; Lindblad-Toh et al. 2000), ligase-independent cloning (de Costa and Tanuri 1998; Rashtchian et al. 1992; Zhou and Hatahet 1995; and commercial kits from Novagen, Invitrogen, BD Biosciences) and Invader assay (Mein et al. 2000).

Normally, these primers are synthesized by phosphoramidite technology and common regions have to be synthesized again and again in different oligonucleotides. It is expensive, especially, when the common part contains a hapten or fluorophore.

This problem becomes evident, for example, in the preparation of sets of padlock probes for SNP-detection projects. Padlock probes are typically 90-120 nt long oligonucleotides, which consist of two locus-specific regions on both 3′ and 5′ ends connected by universal linker part (FIG. 1A). The high price and the low yield of synthesis are the main obstacles for routine usage of padlock probes. Though they were shown to be an excellent tool for SNP detection and in situ localization, only few laboratories work with padlocks until now. Accordingly, the technical problem underlying the present invention was to provide methods for the quantitative and cost-sensitive production of single-stranded nucleic acid molecules that can in particular be employed as padlock probes.

The solution to said technical problem is achieved by providing the embodiments characterized in the claims. Thus, the present invention relates to a method of producing single-stranded nucleic acid molecules from oligo- or polynucleotides wherein each of said oligo- or polynucleotides has a predefined 5′ or 3′ terminus, comprising the steps of (a) annealing an adaptor oligonucleotide simultaneously or step by step to (aa) a first oligo- or polynucleotide; and (ab) a second oligo- or polynucleotide wherein the 5′-terminus of said adaptor oligonucleotide is complementary in sequence to the 5′ terminus of said first oligo- or polynucleotide and the 3′-terminus of said adaptor molecule is complementary in sequence to the 3′ terminus of said second oligo- or polynucleotide; and optionally (a′) simultaneously with or subsequently to step (a) annealing at least one further adaptor oligonucleotide to free termini of said first or second oligonucleotides and to free termini of further oligo- or polynucleotides; (b) optionally filling in gaps between the neighboring ends of said oligo- or polynucleotides; (c) ligating said oligo- or polynucleotides; and (d) removing said at least one adaptor oligonucleotide.

In accordance with the present invention, the term “oligonucleotide” refers to a uni-dimensional (i.e. not branched) stretch of nucleotides, preferably deoxyribonucleotides up to 30 nucleotides. The term also comprises oligonucleotides comprising or consisting totally of ribonucleotides. Also envisaged is that the oligonucleotides comprise unusual nucleotides such as unusual nucleotides as, for example, deoxyuridine, biotinylated or fluorescently labeled nucleotides, spacers or abasic residues. It is preferred that the oligonucleotide employed in the method of the invention consists of the four naturally occurring deoxyribonucleotides, i.e. adenine, cytosine, guanine and tymidine.

The term “polynucleotide” in accordance with the invention may consist of the same types of nucleotides that are described above for oligonucleotides. However, a polynucleotide in accordance with the invention comprises a unidimensional stretch of at least 31 nucleotides.

The term “5′-terminus” refers to the 5′-terminal part of an oligo- or polynucleotide, preferably the terminal 5 or 4 nucleotides.

The term “complementary in sequence” refers to complementarity in sequence of at least 75% of the respective nucleotides, preferably at least 90% of the respective nucleotides and most preferred 100% of the respective nucleotides.

In accordance with the present invention, a novel method of producing oligonucleotides by ligation of individual fragments by a ligase such as T4 DNA ligase is described. It is simple and allows the simultaneous processing of several reactions. The method is quantitative, cheap and does not require individual optimization. The possibility to purify products by HPLC makes the technology suitable for large-scale genomic projects. On the other hand, the same approach may be used for small-scale synthesis of composite primers for two-step PCR amplification and ligation-independent cloning. Small-scale reaction does not require any purification.

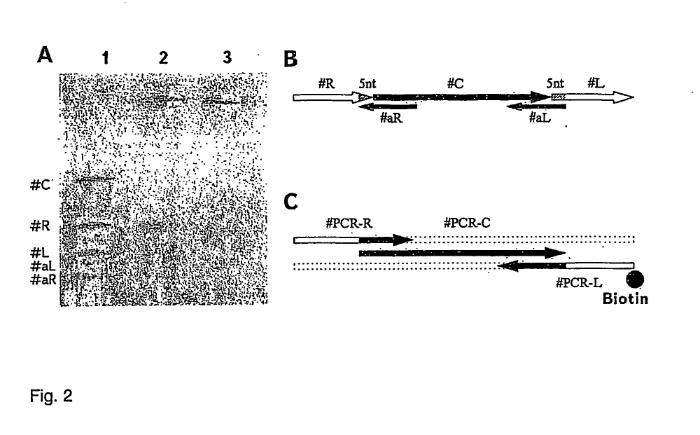

The method of the invention requires oligo- or polynucleotides having a predefined 5′ or 3′ terminus to which an adaptor polynucleotide, which is complementary in sequence to, said predefined 5′ or 3′ terminus is annealed. Simultaneously or subsequently, the second oligo- or polynucleotide is annealed to said adapter oligonucleotide by way of complementarity of its 5′ or 3′ terminus. A schematic overview over the annealing process is provided in FIG. 2B wherein the first oligo- or polynucleotide may be represented by #R, the second oligo- or polynucleotide may be represented by #C and the adaptor oligonucleotide may be represented by #aR. Alternatively, the first oligonucleotide or polynucleotide may be represented by #C, the second oligonucleotide or polynucleotide may be represented by #L and the adaptor oligonucleotide may be represented by #aL.

The situation including optional step (a′) is represented by the complete arrangement of oligonucleotides depicted in FIG. 2B. For example, if #R represents the first oligo- or polynucleotide and #C represents the second oligo- or polynucleotide and #aR represents the adaptor oligonucleotide, then #aL represents the further adaptor oligonucleotide and #L represents the further oligo- or polynucleotide.

If gaps are obtained after annealing of the adaptor oligonucleotide(s) to said first, second and optionally further oligo- or polynucleotide(s) then the gaps are filled in, for example, by polymerase activity such as T4 DNA polymerase activity. Subsequently, the at least two oligo- or polynucleotides are ligated using an appropriate ligase. Appropriate ligases depend, inter alia, on the nature of the oligo- or polynucleotides used for preparation of the single-stranded nucleic acid molecules. For example, if the oligo- or polynucleotides are DNA, than it is preferred to use the T4 DNA ligase. Other ligases, may also be used, for example thermostable commercially available Tth, Taq or Pfu ligases. Another possibility to perform ligation is a chemical template-dependent reaction (Xu and Kool 1999), which uses chemically activated oligonucleotides instead of enzyme.

Finally, the at least one adaptor oligonucleotide is removed. Removal can be effected by denaturated PAGE or chromatography, such as FPLC or HPLC. Other methods are known in the prior art comprising capture of biotine labeled adaptors or destruction of ribonucleotide adaptors by RNase (Nilsson et al. 2000).

The above steps performed in accordance with the present invention per se can be effected by the person skilled in the art according to conventional protocols such as are provided in the appended examples. Temperature ranges include 4 to 42° C. for annealing, fill-in reactions and ligation.

Reaction buffers include conventional reactions buffers such as disclosed, for example, in (Sambrook and Russell 2001).

Preferred is a method of the invention wherein the complementarity in sequence is at least four nucleotides such as 5, 6, 7, 8, 9 or 10 nucleotides. It is particularly preferred that the number of nucleotides which are complementary in sequence is five nucleotides. Also particularly preferred is that there is no mismatch within the stretch of complementarity.

In another preferred embodiment the invention relates to a method wherein annealing and ligation are simultaneously performed. Buffers can easily be adjusted to have annealing and ligation performed simultaneously. If these steps are performed simultaneously, then it is preferred that optional step (b) is omitted. The method of the invention can in this way be accelerated.

It is also preferred in accordance with the method of the present invention that the most valuable oligo- or polynucleotide in step (a) and/or (a′) is provided in molar deficit relative of other oligo- and polynucleotides. The molar deficiency will guarantee that said oligo- or polynucleotide is consumed in the ligation reaction. The term “most valuable oligo- or polynucleotide” with respect to the present invention refers to invention refers to either (i) the most expensive oligonucleotide (labeled by hapten or fluorophore or the longest oligonucleotide) or (ii) oligonucleotide available in less quantity if compared with others.

In another preferred embodiment, the present invention relates to a method, wherein said single-stranded nucleic acid molecules represent a collection of nucleic acid molecules and wherein either said first or said second oligo- or polynucleotide is invariable in sequence between all or essentially all members of said collection of nucleic acid molecules.

The term “essentially all members” refers to at least 90%, preferably at least 95%, more preferred at least 98% and most preferred to at least 99% such as 99.5% or 99.8% of all members.

This advantageous embodiment of the invention relates to, in other terms, a method of producing a collection of single-stranded nucleic acid molecules wherein each member of said collection of nucleic acid molecules comprises a portion that is invariable between all or essentially all members of said collection and at least one portion that is variable between different members of said collection and that is located 5′ or 3′ of said invariable portion, comprising the steps of (a) annealing at least one adaptor oligonucleotide simultaneously or step by step to (aa) an oligo- or polynucleotide representing said invariable portion; and (ab) oligo- or polynucleotides representing said variable portions, wherein (i) a first part of said at least one adaptor oligonucleotide is complementary in sequence to the 5′ terminus of said nucleic acid molecule representing said invariable portion and a second part of the at least one adaptor molecule is complementary in sequence to the 3′ terminus of a nucleic acid molecule representing said variable portion; or (ii) a first part of said at least one adaptor oligonucleotide is complementary in sequence to the 3′ terminus of said nucleic acid molecule representing said invariable portion and a second part of the at least one adaptor molecule is complementary in sequence to the 5′ terminus of a nucleic acid molecule representing said variable portion; (b) optionally filling in gaps between the neighbouring ends of said invariable and said variable portions; (c) ligating the invariable and variable portions; and (d) removing said at least one adaptor oligonucleotide. In accordance with this preferred embodiment, it is further particularly preferred that said nucleic acid molecule representing said invariable portion is annealed with two adapter oligonucleotides, wherein further one of said adapter oligonucleotides is in a first part complementary in sequence with the 5′ end of said nucleic acid molecule representing said invariable portion and the second adapter oligonucleotide is in a first part complementary to the 3′ end of said nucleic acid molecule representing said invariable portion. In this embodiment, the respective termini of the adaptor polynucleotides not annealed to said invariable portion are annealed to termini of oligo- or polynucleotides representing variable portions of the single-stranded nucleic acid molecule. A schematic overview of such an arrangement is provided in FIG. 2B.

This embodiment of the method of the invention is particularly advantageous in the cost-sensitive and easy production, for example, padlock probes. It is also advantageous to use resulting single-stranded nucleic acid molecules in two-step PCR or ligase-independent cloning as will be discussed further below.

In principle, the nucleic acid molecules representing the variable portions may have at least one conserved terminus, namely the terminus that anneals to the adaptor oligonucleotide. In this case adaptor oligonucleotides may be essentially the same for the whole collection. Alternatively, the oligo- or polynucleotides representing variable portions may be without any conservative parts. Then the special adaptor oligonucleotide should be used for annealing of each particular nucleic acid molecule representing the variable portion. The terminus of said special adaptor oligonucleotide not annealed to the nucleic acid molecules representing the invariable portion must be predefined in order to allow a successful annealing reaction.

Most preferred is a method of the invention wherein the further oligo- or polynucleotides are variable in sequence between different members of said collection of nucleic acid molecules.

In accordance with the embodiments pertaining to the production of a collection of single-stranded nucleic acid molecules, it is finally preferred that the 5′ or 3′ termini of said oligo- or polynucleotides representing said variable sequences which anneal to said 5′ or 3′ termini of said adaptor oligonucleotide are invariable between different members of said oligo- or polynucleotides representing said variable sequences.

In another preferred embodiment of the method of the invention, ligation is effected with T4 DNA ligase. Most preferred is that about 1 unit of T4 DNA ligase is reacted in step (c) with about 4 pmol of termini of the oligo- or polynucleotides annealed to said adaptor molecule(s). It is also preferred in this embodiment that the ligation reaction is carried out at a temperature of about 20° C. Ligation efficiency may significantly be increased if the reaction is carried out at, for example, 37° C.—the temperature optimum for T4 DNA ligase. At this temperature, it is required that the complementary sequences comprise 5 or more nucleotides.

Further preferred is a method wherein the ligation reaction is carried out in the presence of some molecular crowding agent, for example, with at least 5% polyethylene glycol. The inclusion of polyethylene glycol above the indicated range is advantageous because it increases the ligation efficiency.

In accordance with this preferred embodiment, it is more preferred that the ligation reaction is carried out in the presence of 12 to 18% polyethylene glycol. It is particularly preferred that the ligation reaction is carried out in the presence of about 15% polyethylene glycol.

Preferred in accordance with the method of the invention is further that said polyethylene glycol is polyethylene glycol 6000.

In an additional preferred embodiment of the present invention, the method further comprises the step of purifying said single-stranded nucleic acid molecules. Purification can be performed according to standard protocols, see for example Sambrook, J., D. Russell. 2001. Molecular cloning: A laboratory manual. Cold Spring Harbor Laboratory, Cold Spring Harbor, N.Y.

Purification advantageously includes PAGE (Polyacrylamide Gel Electrophoresis) preferably under denaturing conditions, FPLC or HPLC or chromatography. Also preferred is an embodiment of the method of the invention further comprising modifying at least one of said oligo- or polynucleotides. In the alternative of the aforementioned embodiment, at least one of said oligo- or polynucleotides is modified when added to the reaction, i.e. the first step of the method of the invention.

In accordance with this preferred embodiment of the invention, the modification of the at least one oligo- or polynucleotide may be effected during one step of the method of the invention, for example when performing the fill-in reaction. Alternatively, a pre-modified oligonucleotide or polynucleotide may be included in the steps of the method of the invention. Modifications may be manifold and include the modifications recited herein below as being preferred.

Advantageously, the modification is a ribonucleotide, a spacer or a nucleotide comprising a detectable label.

Detectable labels include bioluminescent, phosphorescent, biotinilated, fluorescent and radioactive labels such as labels with ³²P or ³H.

In a particularly advantageous embodiment of the method, said oligo- or polynucleotides representing the invariable sequence are modified.

It is also preferred to automate the method of the invention. The ligation reaction may be assembled by liquid handling automated system. It is additionally preferred that the final product is purified by HPLC.

In an additional preferred embodiment of the method of the present invention, said method further comprises employing members of said collection of nucleic acid molecules in ligase-independent cloning (LIC). Composite primers for LIC have gene-specific 3′-parts and special 5′-parts (LIC system, Novagen; In-Fusion PCR cloning, BD Biosciences Clontech; Gateway PCR cloning system, Invitrogen).

The figures show:

FIG. 1: Applications of oligonucleotides with block structure. Gene-specific parts are white, common parts are black. A. Template-dependent ligation of padlock probe. B. Primers for multiplex PCR amplification and ligation-independent cloning. C. Invader assay.

FIG. 2: Padlock synthesis. A. Step by step PAGE analysis of padlock synthesis. Bands were visualized by UV shadowing as described in Methods. Lane 1—unligated primers; lane 2—result of ligation; lane 3—PAGE-purified padlock probe. Aliquots of the same reactions were taken for this gel. B. Scheme of ligation. C. PCR-based approach for padlock synthesis (Antson et al. 2000; Myer and Day 2001). Amplification is performed with two gene-specific primers having long 3′ overhands (#PCR_L and #PCR_R) on template #PCR_C. Single stranded padlock is purified after annealing to Streptavidine paramagnetic particles.

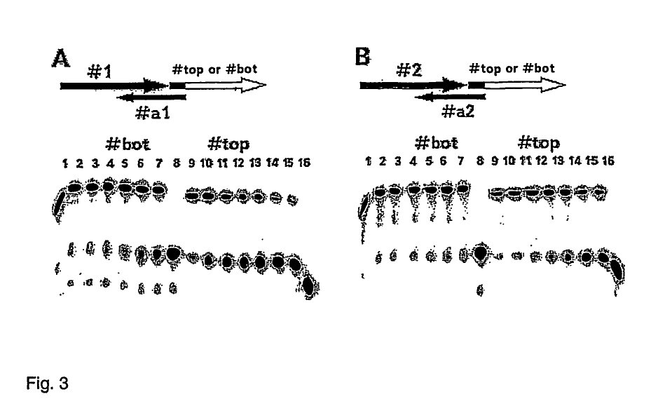

FIG. 3. Ligation of different primers with the same 4 nt overhangs. Ligation of [γ-32P]ATP labeled #top and #bot primers with (#1; #a1) and (#2; #a2); see scheme under Table 1. Ligation was performed as described in Methods. Lanes 1-8: #bot primer; lanes 9-16: #top primer. Lanes (1-7) and (9-15) correspond to the sequential two times dilutions of T4 DNA ligase (400 u for lanes 1 and 9). Lanes 8 and 16—control without ligase. A. Ligation with #1 and #a1. B. Ligation with #2 and #a2.

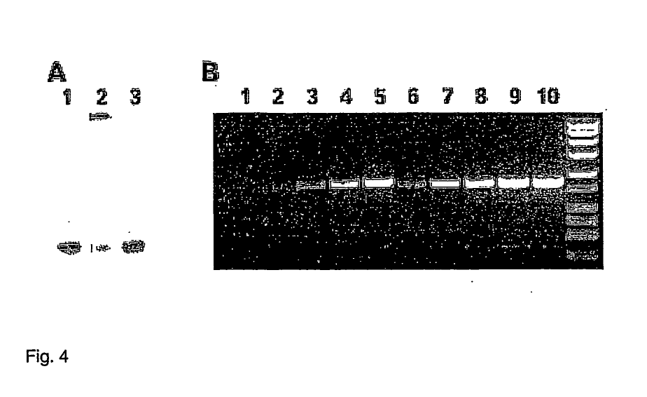

FIG. 4: Application of ligated primers. A. Padlock probe circularizes only in the presence of perfectly matched template. Circular products have decreased mobility in PAGE comparing with linear ones. Lane 1—control without ligase; 2—ligation on #T1 (perfectly matched) template; 3—ligation on #T2 (mismatched) template. B. PCR amplification of ORF of phi29 polymerase (1.7 kb) with composite and gene-specific primers. Lines 1-5—composite primers (#1&#top and #2&#bot); lines 6-10—gene-specific primers (#top and #bot). Lanes, 1 and 6—after 12 cycles; lanes 2 and 7—after 16 cycles; lanes 3 and 8—after 20 cycles; lanes 4 and 9—after 24 cycles; lanes 5 and 10—after 28 cycles. M—marker.

### MATERIALS AND METHODS

Oligonucleotides were synthesized by TIB Molbiol (Berlin, Germany). Primer sequences are given in Table 1. T4 DNA ligase and T4 PNK were from New England BioLabs (Beverly, USA). Tth ligase was from ABgene (UK).

Polyacrylamide gels with radiolabeled oligonucleotides were exposed on a Fuji Imaging Plate without any fixation. For long exposition (more than couple of hours) cassette with gel was freezed at −20° C. Freezing prevents diffusion of even 4 nt long oligonucleotides.

### Padlock Probes.

Phosphorylation of #C and #L oligonucleotides was performed at 37° C. for 1 hour. 1 nmol of primer was incubated in 10 μl of T4 PNK buffer (TrisHCl, pH 7.6 70 mM; MgCl₂ 10 mM; DTT 5 mM) with 1 mM ATP and 2.5 u of T4 PNK flowed by enzyme heat inactivation at 65° C. for 20 minutes. Phosphorylated primers were used in ligation reactions without purification.

The scheme of the ligation-based synthesis of padlock probe is shown on FIG. 2B. 200 pmol-scale ligation reaction was performed for 1 hour at 20° C. in 20 μl of mixture: 1×T4 ligase buffer (TrisHCl, pH 7.5 50 mM; MgCl₂ 10 mM; DTT 10 mM; ATP 1 mM; BSA 25 μg/ml); PEG 6000 15%; T4 DNA ligase 100 u. To ensure, that all #C oligonucleotides are consumed in annealing and ligation reactions, adaptors and locus-specific primers were taken in a slight excess relative to the common primer: #C—200 pmol; #aR=#aL—220 pmol; #R=#L—240 pmol (1:1.1:1.2). In some experiments adaptors were annealed to the common primer before addition of enzyme and locus-specific primers (heating the mixture to 90° C. and cooling gradually to normal temperature), but this measure is not essential for the method of the invention.

Padlock probes may be phosphorylated directly in the ligation mixture after heat inactivation of T4 DNA ligase (e.g. 65° C. for 15 minutes).

Padlocks were purified through denaturing PAGE electrophoresis. The corresponding band was visualized by UV shadowing on the DC Alufolien Kiselgel 60F254 (Merck, Germany) chromatographic plate (or on printer paper with a somewhat lower sensitivity) and was cut out. DNA was ethanol precipitated after elution from the gel in 150 μl of (TrisHCl, pH 7.5 10 mM; EDTA 1 mM; NaCl 200 mM) for 1 hour at 60° C.

Circularization of 40 fmol of a [γ-32P] ATP labeled padlock probe on 2 fmol of matched or mismatched synthetic template was performed in 10 μl of 1×Tth ligation buffer (TrisHCl, pH 8.3 20 mM; MgCl₂ 10 mM; KCl 50 mM; EDTA 1 mM; NAD⁺ 1 mM; DTT 10 mM; Triton X-100 0.1%) by 1 u of Tth ligase for 20 cycles of (94° C. for 20 sec and 60° C. for 3 min).

### Composite Primers for PCR.

Ligation was performed separately for (#top, #1, #a1) and (#bot, #2, #a2) primer sets: 1 hour at 20° C. and 20 min at 70° C. in 10 μl of mixture: 1×T4 ligase buffer; PEG 6000 15%; T4 DNA ligase 400 u; phosphorylated #top or #bot primers—20 pmol; #a1 or #a2—25 pmol; #1 or #2—30 pmol. Composite primers were used in PCR amplification without any purification.

Amplification was performed in 50 μl with Advantage cDNA polymerase Mix (Clontech, USA). 1 μl of phage phi29 suspension (5×10¹⁰ 1/ml; DSMZ, Germany) was used as a template. Parameters of PCR with #top and #bot primers were: 10 pmol of both primers; 96° C. 2 min (95° C. 20 sec, 62° C. 20 sec, 68° C. 1 min 20 sec)×28 cycles. PCR with composite primers: 1 pmol of both composite primers, 10 pmol of #1 and #2 primers; 96° C. 2 min, (95° C. 20 sec, 62° C. 20 sec, 68° C. 1 min 20 sec)×5 cycles, (95° C. 20 sec, 58° C. 20 sec, 68° C. 1 min 20 sec)×23 cycles. Two different annealing temperatures were used for amplification with composite primers because melting temperature of external primer #1 (59.6° C.) was less, than that of internal primers #top and #bot (65° C.).

In this specification, a number of documents is cited. The disclosure content of these documents including manufacturers' manuals, is herewith incorporated by reference in its entirety.

The examples illustrate the invention.

### EXAMPLE 1

### Padlock Synthesis

Padlock probes (padlocks) are typically 90-120 nt oligonucleotides, which consist of two locus-specific regions on both 3′ and 5′ ends connected by universal linker part (FIG. 1A). The ability of padlocks for template-dependent circularization is used for in situ localization and SNP detection (Antson et al. 2000; Lizardi et al. 1998; Myer and Day 2001; Pickering et al. 2002). High quality of locus-specific ends is important in ligation reaction, because they should create a nick with perfect base pairing.

The scheme of the ligation-based synthesis of padlocks is shown on FIG. 2B. Adaptor primers #aL and #aR and central part primer #C (all shown black) are common for the whole set of padlocks. Primers #R and #L (shown white) are locus-specific. 5 nt 3′ and 5′ overhangs of adaptor primers serve for ligation of locus-specific primers. Small excess of adapters (1,1x) and locus-specific primers (1,2x) guarantee that all #C oligonucleotides will be consumed in ligation. The scheme is practically the same for synthesis of oligos with common 3′ or 5′ regions (just one adaptor and one locus-specific primer instead of two).

Melting temperatures of overlaps of adaptor primers with common primer are about 50° C. in the ligation mixture. In principle, it is possible to use shorter #C-complementary segments of adaptor primers thus decreasing the price of the procedure. Overhangs for locus-specific primers were selected to be 5 nt long (see scheme below Table 1). Such a length was satisfactory for the purpose of the present invention. However, longer overhangs showed better ligation efficiency (data not presented). Moreover, for longer overhangs it is possible to increase the ligation efficiency to about twice by carrying out the reaction at 37° C.—i.e. the optimal temperature for T4 DNA ligase.

The method of the invention requires attention to the selection of primer ends (termini) to avoid misligation. In accordance with the present invention it was found that due to the high fidelity of T4 DNA ligase this restriction is not stringent. To estimate the efficiency of mismatch ligation, we have compared ligation of 3′ overhangs (3′-CTCGG . . . 5′) with perfectly matched primers/oligonucleotides (5′- . . . GAGCC-3′) and with two mismatched primers/oligonucleotides: (5′- . . . GAGga-3′) and (5′- . . . GAGCg-3′). In experiments with ten times excess of ligase we have not detected any ligation products for overhangs containing one and two mismatched bases. NQ problems with misligation were observed for overhangs shown in Table 1. It is possible to improve the ligation accuracy by increasing the concentration of monovalent ions in reaction buffer (Wu and Wallace 1989), and to exclude any participation of adaptor oligos in ligation by blocking their 3′ ends.

According to the present New England BioLabs Catalogue, 1 unit of T4 DNA ligase is capable to join about 1.2 pmol of nicks of λ/Hind III digested DNA in 1 hour. In a test ligation of #C with #R and #aR we have obtained a similar result: 1 u-0.4 pmol. Because PEG 6000 increases the ligation efficiency (Pheiffer and Zimmerman 1983), we have checked the influence of 5%-20% PEG 6000 on the reaction. 15% PEG turned out to be the most effective, increasing the ligation efficiency about 15 times: 1 u-6 pmol. For padlock synthesis, 0.5 unit of T4 DNA ligase should be added per 1 pmol of the #C primer. This ratio was determined in a series of small-scale ligations with decreasing amounts of the enzyme (data not shown) and agrees well with titration on individual nicks.

Padlock probes were purified in denaturing PAGE (FIG. 2A). The yield is quantitative: 70% according to spectrophotometer measurements and close to 100% according to PAGE (FIG. 2A). It seems that some UV adsorbing impurities are removed on this step. Purified padlock probes run as single bands in denaturing PAGE (FIG. 2A) and are suitable for SNP-discriminating ligation (FIG. 4A). PAGE purification is the most time-consuming and laborious step of the procedure. However, in conventional phosphoramidite synthesis this step also cannot be avoided (see below). Besides, in ligation-based procedures distinct differences between the length of padlock and initial primers (FIG. 2A) allows to use HPLC purification instead of PAGE. Adaptor primers do not participate in ligation and may be repurified together with padlocks in large-scale projects.

Ligation-based procedure is superior if compared with the conventional phosphoramidite synthesis.

Length restriction. Oligonucleotide-producing companies cannot guarantee a quantitative yield for oligos longer than 100 nt and do not take orders for primers longer than 130 nt. In contrast, the ligation-based method of the present invention is only restricted by the synthesis of individual components: for 3-component padlock probe the procedure is practical up to 150-180 nt.

Price and quality. According to information from all three companies listed in Table 2, a two-step purification (HPLC and PAGE) is required for 80-100 nt long oligonucleotides. HPLC alone cannot separate full-length oligonucleotides from nearby contaminants. Smallest-scale synthesis of one 100 nt oligonucleotide costs about 170 Euro and gives about 3 nmol of product after HPLC purification (Table 2A). Judging from Operon the yield after PAGE purification will be less than 1 nmol.

5 nmol—scale synthesis of single padlock oligonucleotide by ligation method costs 120 Euro (Table 2B). In our hands the yield after PAGE purification is more then 3 nmol. Comparing “170 Euro per 1 nmol” with “120 Euro per 3 nmol”, the ligation-based synthesis of the present invention is four times cheaper. What is more important, the quality of ligase-based synthesis is higher. It provides bands practically without “n−1” contaminants. In principle, HPLC purification may be used instead of PAGE.

Synthesis of large sets. For producing a set of oligonucleotides with block structure by the phosphoramidite technology, common regions have to be synthesized again and again. The price per oligo does not change if compared with the single synthesis. For example, synthesis of 40 100 nt padlocks for 20 SNP loci (two padlocks per loci) by the conventional procedure costs 6720 Euro (Table 2A). In the ligation-based method the price per oligonucleotide drops dramatically, because common parts need to be synthesized only once. The same set costs 1846 Euro. The price difference is 10 fold (6720 Euro, 1 nmol of each padlock; 1846 Euro—3 nmol).

If common parts contain biotin or fluorescein, conventional synthesis will be 2000 Euro more expensive (40 padlocks×50 Euro) and the yield drops about two fold. Ligation-based synthesis will cost 100 Euro more (2 padlocks×50 Euro) and the yield remains the same.

Two PCR based methods of padlock synthesis were suggested recently (Antson et al. 2000; Myer and Day 2001; and FIG. 2C), but they have some disadvantages if compared with proposed ligation-based technology. Locus-specific primers have long overhangs for PCR initiation (˜12 nt (Antson et al. 2000) and ˜18 nt (Myer and Day 2001)). It is impossible to insert modified bases in some definite position during PCR. Using of proofreading polymerase (Antson et al. 2000) results in heterogeneous 3′ ends of padlocks. Finally, single-strand purification by streptavidine PMP's is expensive (about 1 ml of Dynabeads is required for 500 pmol of padlock).

To summarise, the ligation-based technology in accordance with the present invention permits to synthesize parts of composite oligonucleotides in separate reactions of the appropriate scale. Waste is minimal. Adaptor primers may be reused. Synthesis may be automated, because it is compatible with HPLC-based purification. For large sets of composite oligonucleotides the price per one primer tends almost equals the price of locus-specific parts and becomes comparable with that of 40-50 mers, which are widely used now in molecular biology.

### EXAMPLE 2

### Composite Primers for PCR Amplification

Composite primers with gene-specific 3′ regions (FIG. 1B) are used for multiplex PCR amplification (Favis et al. 2000; Lindblad-Toh et al. 2000; Eurogenetics—genome primer sets), ligase-independent cloning (LIC) and Invader assay (Mein et al. 2000). They have “block structure”: 3′ parts (17-25 nt) identify gene target and 5′ regions define some particular application: second-stage PCR (16-20 nt), concrete LIC scheme (LIC system, Novagen—15 nt; In-Fusion PCR cloning, BD Biosciences Clontech—16 nt; Gateway PCR cloning system, Invitrogen—29 nt) and so on.

Some applications require combination of blocks. For example, the same gene-specific part should be attached to different vector-specific parts for optimization of the protein expression (Dieckman et al. 2002). Frequently, the required amount of primers is very small. Only one successful PCR reaction is necessary for LIC or for preparation of genome-sequencing tags (Eurogenetics: http://www.eurogentec.com/code/en/geno_dnaa_prod.htm).

Each composite primer should be synthesized individually by conventional phosphoroamidite technology. Moreover, decreasing of the synthesis scale does not decrease the price of the primers proportionally. Some LIC methods require insertion of modified bases in composite oligonucleotides, for example 4-5 dUracils (Rashtchian et al. 1992) or 2-3 phosphorothioate bonds (de Costa and Tanuri 1998; Zhou and Hatahet 1995). In this case the price of composite oligos increases in 2-3 times.

PCR amplification is more difficult with long composite primers. Sometimes, the only way to obtain single-band product is to use cloned or preliminarily amplified fragments as a template. This means that two sets of primers should be used in this case: gene-specific pair and composite pair.

It is very convenient to prepare small amounts of composite primers by attaching presynthesized blocks to each other. In this case it is possible to combine gene-specific parts with different 5′ parts.

Small-scale procedure for preparation of composite primers from individual blocks with the help of T4 RNA ligase was described previously (Kaluz et al. 1995). This enzyme needs no overhangs for joining of two oligos. The only disadvantage of the method is that some part of the reaction products (depends on excess of external primer) have duplication of gene-specific 3′ part. It is not critical for some applications, but can provide problems for cloning. Another disadvantage is that phosphorylation and ligation should be performed separately.

In contrast to the above recited prior art approaches, the (T4 DNA) ligase based method of the present invention requires overhangs for ligation, but guarantees the homogeneity of the product. Ligated primers may be used in PCR without any purification because PEG and other components of ligation mixture do not inhibit PCR. Ligation and phosphorilation may be performed simultaneously in a ligation buffer (result not shown).

Adaptor primers do not interfere with PCR. To decrease the risk of false priming by adaptor primers and mis-annealing of long composite primers it is most appropriate to use a mixture of external and composite primers (in molar ratio 10:1) instead of composite primers alone in an amplification reaction.

An example of amplification with composite primers produced by ligation is shown in FIG. 4B. The ORF of phi 29 polymerase (Blanco and Salas 1996) was amplified from 1 μl of phage culture. In both cases the required products were obtained. PCR was slower with composite primers if compare with gene specific ones. Worse kinetics at least partly depends on external primers, because the amplification is more effective with (#top and #bot) pair than with (#1 and #2) pair (results not shown).

There are two possible schemes of design of adaptor primers for ligation. One approach is to order individual adaptor for each particular combination of 5′ and 3′ parts. Adaptors should be 10-14 nt long (two 5-7 nt overlaps). They are cheaper than long composite primers. The price of external primers should be left out of account, because they may be used with a number of gene-specific primers. A 20 pmol aliquot of HPLC purified external primer costs less then 10 Euro-cent

Another approach is to use the standard adaptors and to prepare gene-specific primers with predefined overhangs (as in the method for padlock preparation, see above). Long overhangs may conflict with some cloning schemes, but too short overhangs may be a bar for the effective ligation. G/C-reach 5 nt overhangs (5′- . . . GAGCC-3′) and (5′-ACGGG . . . -3′) are sufficient for effective ligation (see padlock preparation above). To obtain an idea about the suitability of shorter sequences we have tried to use the (5′-TATG . . . -3′) sequence for the preparation of composite primers. This sequence was selected because ATG may be combined with the first Met codon of the open reading frame. It turns out, that 4 nt A/T-rich overhang requires much more ligase. Moreover, different amounts of enzyme were necessary for different combinations of primers (FIG. 3). Nevertheless, 1 μl (400 u) of T4 ligase was enough to prepare 20 pmol of composite primers. It costs about 1 Euro, much less, if compared with the price of long composite primers.

### CONCLUSION

Ligation-based technology is based on construction of composite oligonucleotides from individual blocks. Here we have demonstrated, how this method may be applied for two different tasks:
(i) preparative-scale production of padlock probes;
(ii) small-scale synthesis of PCR primers.

Preparative-scale procedure gives products of high quality. In contrast to the conventional phosphoroamidite synthesis, HPLC may be used instead of PAGE purification.

Small-scale procedure is fast one-tube reaction. It does not require any purification and may be automated.

In both cases the suggested method is considerably cheaper if compared with the conventional phosphoramidite synthesis. The price difference increases at least two fold, when oligonucleotides contain modified bases.

### REFERENCES

Antson, D. O., A. Isaksson, U. Landegren, M. Nilsson. 2000. PCR-generated padlock probes detect single nucleotide variation in genomic DNA. Nucleic Acids Res. 28(12): e58.
Blanco, L., M. Salas. 1996. Relating structure to function in phi29 DNA polymerase. J Biol Chem. 271(15):8509-8512.
da Costa, L. J., A. Tanuri. 1998. Use of T7 gene 6 exonuclease and phosphorothioated primers for the manipulation of HIV-1 infectious clones. J Virol Methods. 72(1):117-21.
Dieckman, L., M. Gu, L. Stols, M. I. Donnelly, F. R. Collart. 2002. High throughput methods for gene cloning and expression. Protein Expr Purif. 25(1):1-7.

Favis, R., J. P. Day, N. P. Gerry; C. Phelan, S. Narod, F. Barany. 2000. Universal DNA array detection of small insertions and deletions in BRCA1 and BRCA2. Nat Biotechnol. 18:561-564.
Kaluz, S., M. Kaluzova, A. P. Flint. 1995. Enzymatically produced composite primers: an application of T4 RNA ligase-coupled primers to PCR. Biotechniques. 19(2):182-4, 186.
Lindblad-Toh, K., E. Winchester, M. J. Daly, D. G. Wang, J. N. Hirschhorn, J. P. Laviolette, K. Ardlie, D. E. Reich, E. Robinson, P. Sklar, N. Shah, D. Thomas, J. B. Fan, T. Gingeras, J. Warrington, N. Patil, T. J. Hudson, E. S. Lander. 2000. Large-scale discovery and genotyping of single-nucleotide polymorphisms in the mouse. Nat Genet. 24:381-386.
Lizardi, P. M., X. Huang, Z. Zhu, P. Bray-Ward, D. C. Thomas, D. C. Ward. 1998. Mutation detection and single-molecule counting using isothermal rolling-circle amplification. Nat Genet. 19:225-232.
Mein, C. A., B. J. Barratt, M. G. Dunn, T. Siegmund, A. N. Smith, L. Esposito, S. Nutland, H. E. Stevens, A. J. Wilson, M. S. Phillips, N. Jarvis, S. Law, M. de Arruda, J. A. Todd. 2000. Evaluation of single nucleotide polymorphism typing with invader on PCR amplicons and its automation. Genome Res. 10:330-343.
Myer, S. E., D. J. Day. 2001. Synthesis and application of circularizable ligation probes. Biotechniques. 30:584-593.
Nilsson, M., G. Barbany, D-O. Antson, K. Gertow, U. Landegren. 2000. Enhanced detection and distinction of RNA by enzymatic probe ligation. Nature biotechnology. 18:791-793
Pheiffer, B. H., S. B. Zimmerman. 1983. Polymer-stimulated ligation: enhanced blunt- or cohesive-end ligation of DNA or deoxyribooligonucleotides by T4 DNA ligase in polymer solutions. Nucleic Acids Res. 11:7853-7871.
Pickering, J., A. Bamford, V. Godbole, J. Briggs, G. Scozzafava, P. Roe, C. Wheeler, F. Ghouze, S. Cuss. 2002. Integration of DNA ligation and rolling circle amplification for the homogeneous, end-point detection of single nucleotide polymorphisms. Nucleic Acids Res. 30:e60.
Rashtchian, A., G. W. Buchman, D. M. Schuster, M. S. Berninger. 1992. Uracil DNA glycosylase-mediated cloning of polymerase chain reaction-amplified DNA: application to genomic and cDNA cloning. Anal Biochem. 206(1):91-7.
Sambrook, J., D. Russell. 2001. Molecular cloning: a laboratory manual. Cold Spring Harbor Laboratory. Cold Spring harbor, N.Y.
Wu, D. Y., R. B. Wallace. 1989. Specificity of the nick-closing activity of bacteriophage T4 DNA ligase. Gene. 76:245-254.
Xu, Y., E. T. Kool. 1999. High sequence fidelity in a non-enzymatic DNA autoligation reaction. Nucleic Acid Res. 27(3):875-81.
Zhou, M., Z. Hatahet. 1995. An improved ligase-free method for directional subcloning of PCR amplified DNA. Nucleic Acids Res. 23(6):1089-90.

### Tables

TABLE 1
Oligonucleotide sequences.

SNP positions in template primers (T1 and T2) are in bold.
Alignments for ligation-based synthesis of padlock and PCR
primers are shown under the table. Overhangs for ligation
are in bold.

| | |
|---|---|
| #C | GGAGGTTGCGAGGCGTATTCATTGCTCAGAATTCACGACTCACG |
| #aR | CCTCGCAACCTCCGGCTC |
| #aL | CCCGTCGTGAGTCGTGAATT |
| #R | TTGTAAAACGTCGGGAGAAACAGAGAGCC |
| #L | ACGGGACATTTAAGACCAAACTG |
| #T1 | CTCTCTGTTTCTCCCGACGTTTTACAACAGTTTGGTCTTAAATGTTCGCCGC |
| #T2 | TCTCTGTTTCTCCCGACGTTTTACAATAGTTTGGTCTTAAATGTTCGCCG |
| #top | TATGAAGCATATGCCGAGAAAGATG |
| #bot | TATGTTTGATTGTGAATGTGTCATCAAC |
| #1 | TTTTGTTTAACTTTAAGAAGGAGATATACA |
| #2 | GATCCTCAGTGGTGGTGGTGGTGGTGCA |
| #a1 | CATATGTATATCTCCTTCTTA |
| #a2 | CATATGCACCACCA |

Allignment of oligonucleotides for preparation of padlock:

| | |
|---|---|
| #R | 5′-TTGTAAAACGTCGGGAGAAACAGAGAGCC-3′ |
| #L | 5′-ACGGGACATTTAAGACCAAACTG-3′ |
| #C | 5′-GGAGGTTGCGAGGCGTATTCATTGCTCAGAATTCACGACTCACG-3′ |
| #aR | 3′-CTCGGCCTCCAACGCTCC-5′ |
| #aL | 3′-TTAAGTGCTGAGTGCTGCCC-5′ |

TABLE 2
Synthesis of 100 nt primer.
(A) Conventional method.

| | Synthesis | Price | | Guaranteed |
|---|---|---|---|---|
| Company | scale | per base | Purification | yield and price |
| Operon | 1 μmol | 2.05 | HPLC&PAGE | 0.5 nmol |
| | | Euro | (118 Euro) | HPLC&PAGE |
| | | | | 323 Euro |
| MWG | >0.2 μmol | 1.78 | HPSF included | 3 nmol |
| | | Euro | | HPSF* |
| | | | | 178 Euro |
| TIB | >0.2 μmol | 1.68 | HPLC included | 3 nmol |
| | | Euro | | HPLC* |
| | | | | 168 Euro |

(B) Ligation-based method
(prices are given according to TIB).

| | | Guaranteed yield and |
|---|---|---|
| Component | Scale and purification | price |
| #L, #R | up to 30 bases long, HPLC purified | 5 nmol; 39.08 Euro |
| #aL, #aR | up to 20 bases long, standard | 5 nmol; 19.98 Euro |
| | purification | |
| #C | up to 60 bases long, standard | 5 nmol; 55.00 Euro |
| | purification | |
| T4 DNA | 1250 u | 3.25 Euro |
| ligase | | |
| T4 PNK | 25 u | 2.50 Euro |
| Total: | | 5 nmol |
| | | 120 Euro |

*both companies recommend to perform additional PAGE purification in laboratory.
Operon: QIAGEN Operon GmbH, Cologne, Germany;
MWG: MWG Biotech, Ebersberg, Germany
TIB: TIB MOLBIOL, Berlin, Germany

TABLE 3
Ligation-based synthesis of 40 100 nt padlocks for 20 SNP loci
(two padlocks per one locus).

| | | Price per 20 loci | |
|---|---|---|---|
| Component | Scale and purification | (Euro) | |
| #L1, #L2, #R | 5 nmol of each, HPLC purified | 1172 | |
| #aL and #aR | 500 nmol of each*, standard | 114 | |
| | purification | | |
| #C1, #C2 | 250 nmol of each*, standard | 330 | |
| | purification | | |
| T4 DNA ligase | 50000 u | 130 | |
| T4 PNK | 1000 u | 100 | |
| Total: | | 1846 | Euro |
| Per one | 5 nmol | 46.2 | Euro |
| padlock: | | | |

*Two times more, than is required for 40 padlocks.

1. A method of producing single-stranded nucleic acid molecules from oligo- or polynucleotides wherein each of said oligo- or polynucleotides has a predefined 5′ or 3′ terminus, comprising the steps of
(a) annealing an adaptor oligonucleotide simultaneously or step by step to
(i) a first oligo- or polynucleotide; and
(ii) a second oligo- or polynucleotide,
wherein the 5′-terminus of said adaptor oligonucleotide is complementary in sequence to the 5′ terminus of said first oligo- or polynucleotide and the 3′-terminus of said adaptor molecule is complementary in sequence to the 3′ terminus of said second oligo- or polynucleotide; and
(b) simultaneously with or subsequently to step (a) annealing at least one further adaptor oligonucleotide to free termini of said first or second oligonucleotides and to free termini of further oligo- or polynucleotides;
(c) optionally filling in gaps between the neighbouring ends of said oligo- or polynucleotides;
(d) ligating said oligo- or polynucleotides; and
(e) removing said at least one adaptor oligonucleotide.

2-22. (canceled)

23. A method of making a single stranded nucleic acid molecule comprising:
(a) providing a first oligonucleotide;
(b) providing a second oligonucleotide;
(c) annealing a first adaptor oligonucleotide to said first and said second oligonucleotide, wherein the 5′ terminus of said adaptor oligonucleotide is complementary to the 5′ terminus of said first oligonucleotide and the 3′ terminus of said adaptor oligonucleotide is complementary to the 3′ terminus of said second oligonucleotide;
(d) ligating said first and second oligonucleotides; and
(e) removing said adaptor oligonucleotide.

24. The method of claim 23, further comprising the step of annealing a second adaptor oligonucleotide to the free termini of said first or second oligonucleotides.

25. The method of claim 24, further comprising the step of annealing a third oligonucleotide to said second adaptor oligonucleotide.

26. The method of claim 23, further comprising the step of filling in a gap between the neighboring ends of said first and second oligonucleotides.

27. The method of claim 23, wherein said adaptor oligonucleotide comprises at least four consecutive nucleotides that are complementary to said first and said second oligonucleotides.

28. The method of claim 23, wherein said annealing and ligating steps are simultaneous.

29. The method of claim 27, wherein said annealing and ligating steps are simultaneous.

30. The method of claim 23, wherein said first adaptor is provided in molar excess of said first or second oligonucleotides.

31. The method of claim 23, wherein said single stranded nucleic acid is a member of a collection of single stranded nucleic acids and either said first or second oligonucleotide is invariable in sequence between all members of said collection.

32. The method of claim 31, wherein either said first or said second oligonucleotide, which is not invariable, is variable in sequence between different members of said collection.

33. The method of claim 31, wherein said oligonucleotides are variable in sequence between different members of said collection of nucleic acid molecules.

34. The method of claim 32, wherein the oligonucleotide comprising a variable sequence is in molar excess over said oligonucleotide that comprises an invariable sequence.

35. The method of claim 32, wherein the 5′ or 3′ termini of said oligonucleotide that is variable in sequence, which anneal to said 5′ or 3′ termini of said adaptor oligonucleotide, are invariable between different members of said oligonucleotides of variable sequences.

36. The method of claim 23, wherein said ligating step comprises T4 DNA ligase.

37. The method of claim 23, wherein said ligating step comprises 5% polyethylene glycol.

38. The method of claim 23, wherein said ligating step comprises 15% polyethylene glycol.

39. The method of claim 23, wherein said ligating step comprises polyethylene glycol 6000.

40. The method of claim 23, wherein said ligating step comprises reacting about 1 unit of T4 DNA ligase with about 4 pmol of termini of said oligonucleotides that are annealed to said adaptor oligonucleotide.

41. The method of claim 23, further comprising purifying said single stranded nucleic acid molecules.

42. The method of claim 41, wherein said purifying step comprises PAGE electrophoresis, HPLC, or chromatography.

43. The method of claim 23, further comprising modifying at least one oligonucleotide.

44. The method of claim 23, wherein at least one oligonucleotide is modified.

45. The method of claim 44, wherein said modification is a ribonucleotide, a spacer, or a nucleotide comprising a detectable label.

46. The method of claim 31, wherein said oligonucleotide comprising an invariable sequence is modified.

47. The method of claim 31, further comprising the identification of an SNP.

48. The method of claim 31, wherein said members of said collection of nucleic acid molecules are used in a ligase-independent cloning or two-step PCR.


SEQUENCE LISTING (13 sequences)

SEQ ID NO: 1 - DNA, 44 bp, Artificial Sequence
  Synthetic oligonucleotide sequence
ggaggttgcgaggcgtattcattgctcagaattcacgactcacg

SEQ ID NO: 2 - DNA, 18 bp, Artificial Sequence
  Synthetic oligonucleotide sequence
cctcgcaacctccggctc

SEQ ID NO: 3 - DNA, 20 bp, Artificial Sequence
  Synthetic oligonucleotide sequence
cccgtcgtgagtcgtgaatt

SEQ ID NO: 4 - DNA, 29 bp, Artificial Sequence
  Synthetic oligonucleotide sequence
ttgtaaaacgtcgggagaaacagagagcc

SEQ ID NO: 5 - DNA, 23 bp, Artificial Sequence
  Synthetic oligonucleotide sequence
acgggacatttaagaccaaactg

SEQ ID NO: 6 - DNA, 52 bp, Artificial Sequence
  Synthetic oligonucleotide sequence
ctctctgtttctcccgacgttttacaacagtttggtcttaaatgttcgccgc

SEQ ID NO: 7 - DNA, 50 bp, Artificial Sequence
  Synthetic oligonucleotide sequence
tctctgtttctcccgacgttttacaatagtttggtcttaaatgttcgccg

SEQ ID NO: 8 - DNA, 25 bp, Artificial Sequence
  Synthetic oligonucleotide sequence
tatgaagcatatgccgagaaagatg

SEQ ID NO: 9 - DNA, 28 bp, Artificial Sequence
  Synthetic oligonucleotide sequence
tatgtttgattgtgaatgtgtcatcaac

SEQ ID NO: 10 - DNA, 30 bp, Artificial Sequence
  Synthetic oligonucleotide sequence
ttttgtttaactttaagaaggagatataca

SEQ ID NO: 11 - DNA, 28 bp, Artificial Sequence
  Synthetic oligonucleotide sequence
gatcctcagtggtggtggtggtggtgca

SEQ ID NO: 12 - DNA, 21 bp, Artificial Sequence
  Synthetic oligonucleotide sequence
catatgtatatctccttctta

SEQ ID NO: 13 - DNA, 14 bp, Artificial Sequence
  Synthetic oligonucleotide sequence
catatgcaccacca